(19) United States
(12) Patent Application Publication
(10) Pub. No.: US 2024/0382338 A1
(43) Pub. Date: Nov. 21, 2024

(54) HAND WARMER

(71) Applicant: Shenzhen Zheyi Technology Co., Ltd., Shenzhen (CN)

(72) Inventor: Guolong GAO, Shenzhen (CN)

(21) Appl. No.: 18/201,191

(22) Filed: May 24, 2023

(30) Foreign Application Priority Data
May 17, 2023 (CN) 2023211972374

(51) Int. Cl. A61F 7/00

(52) U.S. Cl. CPC A61F 7/007; A61F 2007/0036

(57) ABSTRACT

A hand warmer includes a first body and a second body with the same structure as the first body and mutually combined with the first body, each of the first and second bodies including a long-strip-shaped housing that includes a heating surface and a contacting surface opposite to the heating surface, and a heat generating unit received in the housing; a plurality of first inserting members arranged on the contacting surface of the first body, and a plurality of second inserting members arranged on the contacting surface of the second body and correspondingly connected to the plurality of first inserting members; after the first inserting member is engaged with the second inserting member, both the first body and the second body are combined into a whole, and the first body and the second body can be separated from a connection position between the first body and the second body.

### BACKGROUND

### 1. Technical Field

The present disclosure generally relates to the technical field of hand warming equipments, and especially relates to a hand warmer that can be combined or used separately.

### 2. Description of Related Art

A hand warm device, such as a hand warmer, is a device with a small volume that can be held in hands for warming hands, which includes a heating circuit therein, so that the hand warmer is held by hands by touching or holding a surface of the hand warmer for warming hands. In this way, it is particularly suitable for using the hand warmer in cold areas or indoors or outdoors. At the same time, the hand warmer is also small and convenient portability.

The hand warmer can be sold individually or configured as left and right hand kits to be sold. A single hand warmer can only provide warmth for one hand, such as a left hand or a right hand, while the left and right hand warmer kits are a combination of two hand warmers that are sold for using by left and right hands, so that the left and right hand warmer kits can be respectively held by the left hand and the right hand, to warm both hands simultaneously.

However, a conventional left and right hand warmer kits are difficult to be received due to a fact that the two hand warmers are separated and can't be combined together for only warming one hand.

Therefore, a new hand warmer should be developed in order to solve the problems shown above.

### SUMMARY

The technical problems to be solved: in view of the shortcomings of the related art, the present disclosure provides a hand warmer that can be combined or used separately, which can combine a first body and a second body together for using by one hand, or separately use by left and right hands, respectively, and a combination of the first and second bodies is easily received and carried.

The technical solution adopted for solving technical problems of the present disclosure is:

A hand warmer that can be combined or used separately includes:
a first body and a second body with the same structure as the first body, and mutually combined with the first body; and wherein
each of the first body and the second body includes a long-strip-shaped housing and a heat generating unit received in the housing, the housing including a heating surface arranged on an outer surface thereof, and a contacting surface opposite to the heating surface; and wherein the heat generating unit is configured to generate heat and then transmit the heat to the heating surface of the housing, and the heating surface of the housing is in a curved shape suitable for being gripped by a hand;
the first body including a plurality of first inserting members arranged on the contacting surface of the housing;
the second body including a plurality of second inserting members arranged on the contacting surface of the housing and correspondingly connected to the plurality of first inserting members; and wherein
after the plurality of first inserting members is engaged with the plurality of second inserting members, correspondingly, both the first body and the second body are combined into a whole, and the first body and the second body can be separated from a connection position between the first body and the second body.

Wherein the first inserting member is an inserting post protruding outwardly from the contacting surface, and the second inserting member is a hole concavely arranged on the contacting surface and engaged with the first inserting member; or the first inserting member is a hole concavely arranged on the contacting surface, and the second inserting member is an inserting post protruding outwardly from the contacting surface and engaged with the first inserting member.

Wherein a front side surface of the housing is the heating surface, and a back side surface of the housing is the contacting surface; after aligning and pressing the contacting surface of the first body with the contacting surface of the second body, the plurality of first inserting members is connected to the plurality of second inserting members in a corresponding manner, and the contacting surface of the first body is in contact with the contacting surface of the second body.

Wherein a range of a total thickness of the first body and the second body is 20-40 mm after the first body is engaged with the second body.

Wherein the housing includes a front housing, a back housing arranged behind the front housing and connected to the front housing, and a heat conducting shell arranged in front of the front housing and connected to the front housing, a front side surface of the heat conducting shell is the heating surface, and a back side surface of the back housing is the contacting surface.

Wherein the back side surface of the back housing is a plane.

Wherein a middle part of the back side surface of the back housing is a plane, and two ends of the back side surface of the back housing are curved surfaces.

Wherein the heat generating unit includes a temperature sensor, and a fixing member is arranged on the front housing or the heat conducting shell and configured to fix with the temperature sensor and be in contact with the heat conducting shell.

Wherein the front housing includes a wiring window passing through the front housing from front and back, and the fixing member includes a rod, a clamping groove arranged on the rod and facing the heat conducting shell, and a wiring groove connected with the clamping groove and passing through the rod from front and back; the rod including a first end connected to the front housing, and a second end opposite to the first end and suspended inside the wiring window.

Wherein an installation portion is arranged on a surface of the rod towards the heat conducting shell, and connected to both the clamping groove and the wiring groove.

Wherein a flange is arranged on an edge of the heat conducting shell, and a snapping groove is arranged on a surface of the front housing facing the heat conducting shell and clamped with the flange.

Wherein a plurality of protrusions is spaced in the clamping groove of the front housing and abutting against the flange.

Wherein a screw is hidden in the hole of the contacting surface; and/or
the inserting post on the contacting surface is a tubular post, and the screw is hidden and arranged inside the tubular post.

Wherein the heat generating unit includes a control circuit board electrically connected to the temperature sensor, a battery and a heat generating member electrically connected to the control circuit board, respectively; both the control circuit board and the battery arranged between the front housing and the back housing, and the heating generating member installed between the front housing and the heat conducting shell and being in contact with the heat conducting shell.

Wherein the heat generating member is a PI flexible electrothermal film attached to the heat conducting shell.

Wherein a high-temperature resistant foam is arranged between the PI flexible electrothermal film and the front housing.

Wherein a charging interface is arranged on the contacting surface of the housing and electrically connected with the heating control circuit.

Wherein the housing is further provided with a control switch electrically connected with the heating control circuit.

Wherein the housing is further provided with an LED signal lamp electrically connected with the heating control circuit.

It should be understood that within the scope of the present disclosure, the above technical features of the present disclosure and technical features specifically described in the following (such as embodiments of the present disclosure) can be combined with each other to form a new or preferred technical solution, which will not be described here one by one due to space limitations.

The present disclosure provides the advantages as below.

First, the first body and the second body can be combined together and used separately to meet various usage requirements.

After the first inserting member of the first body is connected to the second inserting member of the second body, both the first body and the second body are combined into a whole, at this time, the hand warmer can be used by one hand to hold a combination of the first and second bodies of the hand warmer; when a user needs to warm left and right hands separately, the first body and the second body are separated from each other to be used by left and right hands, respectively. That is, the hand warmer of the present disclosure can be used by one hand after combining the first and second bodies, or can use one of the first and second bodies by one hand after separating the first body from the second body; the first and second bodies that have been separated can be held to be used by each of left and right hands, respectively, or only be held to be used by one hand, rather than using by the other hand, so as to adapt to different usage scenarios and requirements.

Second, a combination operation of the hand warmer is convenient and a connection structure of the hand warmer that is processed and manufactured is simple, and a cost is low.

When combining the first and second bodies, the contacting surfaces of the first and second bodies are aligned and pressed, so as to insert the first and second inserting members together; at the same time, the insertion and connection structure is simple to be processed, and compared to other connection structures, such as a buckle connection and a magnetic suction connection, the cost is lower.

Third, the hand warmer of the present disclosure is easily received and carried.

When the hand warmer of the present disclosure is not used, both the first body and the second body are combined with each other to form a fixed whole, which can reduce a space occupation and be conveniently received and carried.

### BRIEF DESCRIPTION OF THE DRAWINGS

In order to more clearly understand the technical solution hereinafter in embodiments of the present disclosure, a brief description to the drawings used in detailed description of embodiments hereinafter is provided thereof. Obviously, the drawings described below are some embodiments of the present disclosure, for one of ordinary skill in the related art, other drawings can be obtained according to the drawings below on the premise of no creative work.

FIG. 1 is a schematic view of a hand warmer that can be combined or used separately in accordance with a first embodiment of the present disclosure.

FIG. 2 is similar to FIG. 1, but shown a first body separated from a second body of the hand warmer of FIG. 1.

FIG. 3 is a schematic view of a contacting surface of the first body aligned with a contacting surface of the second body in a separate state of the hand warmer of FIG. 1.

FIG. 4 is an assembly view of the first body engaged with the second body of the hand warmer of FIG. 3.

FIG. 5 is an exploded, schematic view of the first and second bodies of the hand warmer of FIG. 1.

FIG. 6 is a schematic view of the first body of the hand warmer of FIG. 3, shown two ends of the contacting surface of the first body is curved surfaces.

FIG. 7 is an exploded, schematic view of a heat conducting shell and a front housing of the first body of the hand warmer of FIG. 1.

FIG. 8 is a schematic view of the second body of the hand warmer of FIG. 1, after removing the heat conducting shell, a heat generating member, a high-temperature resistant foam, and a temperature sensor thereof.

FIG. 9 is an enlarged view of a circle A of FIG. 8.

FIG. 10 is a schematic view of the heat conducting shell of the hand warmer of FIG. 1.

FIG. 11 is a schematic view of the first and second bodies with screws received therein of the hand warmer of FIG. 1.

FIG. 12 is a schematic diagram of circuits of the hand warmer of FIG. 1.

FIG. 13 is a schematic view of a hand warmer that can be combined or used separately in accordance with a second embodiment of the present disclosure.

FIG. 14 is similar to FIG. 13, but shown that the first body is separated from the second body thereof.

The member labels according to the exemplary embodiment of the present disclosure shown as below:
100 hand warmer, 1 first body, 2 second body, 3 housing, 301, 331 front side surface, 302, 321 back side surface, 31 front housing, 311 wiring window, 312 installation window, 32 back housing, 3211 middle part, 3212 end, 33 heat conducting shell, 332 edge, 34 flange, 341 clamping strip, 35 snapping groove, 351 protrusion, 4 heat generating unit, 41 temperature sensor, 42 control circuit board, 43 battery, 44 heat generating member, 441 PI flexible electrothermal film, 5 heating surface, 6 contacting surface, 7 first inserting member, 8 second inserting member, 91 inserting post, 910 tubular post, 92 hole, 10 fixing member, 101 rod, 1011 first end, 1012 second end, 1013 surface, 102 clamping groove, 103 wiring groove, 104 installation portion, 11 screw, 12 high temperature resistant foam, 13 charging interface, 14 control switch, 15 LED signal lamp.

### DETAILED DESCRIPTION

Reference will now be made in detail to embodiments, examples of which are illustrated in the accompanying drawings. In the following detailed description, numerous specific details are set forth in order to provide a thorough understanding of the subject matter presented herein. Obviously, the implementation embodiment in the description is a part of the present disclosure implementation examples, rather than the implementation of all embodiments, examples. According to the described embodiment of the present disclosure, all other embodiments obtained by one of ordinary skill in the related art on the premise of no creative work are within the protection scope of the present disclosure.

In the description of the present disclosure, it needs to be explained that all the directional indicators (such as the terms: “upper”, “below”, “left”, “right”, “front”, “back” . . . ), are shown in the specification of the present disclosure. The indicated orientation or position of the terms shown in the detailed description is based on the orientation or position shown in the figures of the accompanying drawings of the present disclosure, which is only to easily simplify the description of the present disclosure, but not indicated that the devices or members of the present disclosure should have a particular orientation or should be designed and operated in a particular orientation. So the terms illustrated in the detail description are not by way of the limitation of the present disclosure.

Furthermore, in the description of the present disclosure, the terms such as “first” and “second” shown in the specification are only used to describe, but not indicated that the members of the present disclosure is important or represented the amount of the members. That is, the features limited by the terms of “first” and “second” may explicitly or implicitly include one or more features.

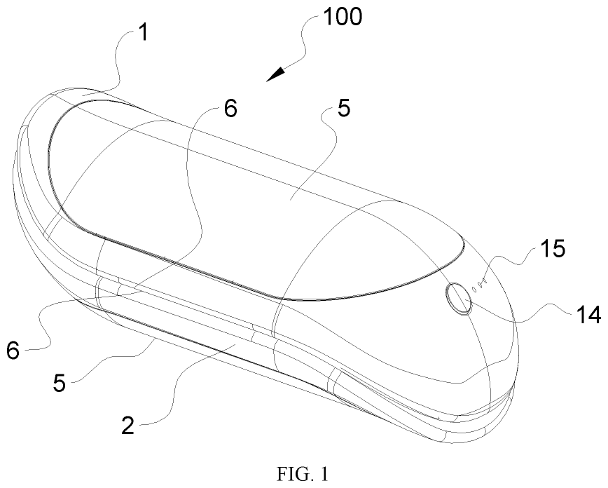

Referring to FIGS. 1-5, a hand warmer 100 that can be combined or used separately in accordance with a first embodiment of the present disclosure includes a first body 1 and a second body 2 with the same structure as the first body 1 and mutually combined with the first body 1 that can be held by human hands. The first body 1 and the second body 2 can be used independently, and can be held by left and right hands, respectively, so as to independently warm left and right hands. The first body 1 and the second body 2 are basically the same structure and can be combined with each other. A heat generating unit 4 is arranged inside each of the first body 1 and the second body 2, to generate heat, so that a person's left hand can hold the first body 1 for warming the left hand, while the right hand can hold the second body 2 for warming the right hand.

Specifically, each of the first body 1 and the second body 2 includes a long-strip-shaped housing 3 and a heat generating unit 4 received in the housing 3. The housing 3 includes a heating surface 5 arranged on an outer surface thereof, and a contacting surface 6 opposite to the heating surface 5. The heat generating unit 4 is equipped with heating elements, such as heating wires and heating plates, etc, so that heat generated by the heating elements is transmitted to the heating surface 5 of the housing 3, and hands of a person can contact the heating surface 5 on the outer surface of the housing 3 for warming the hands.

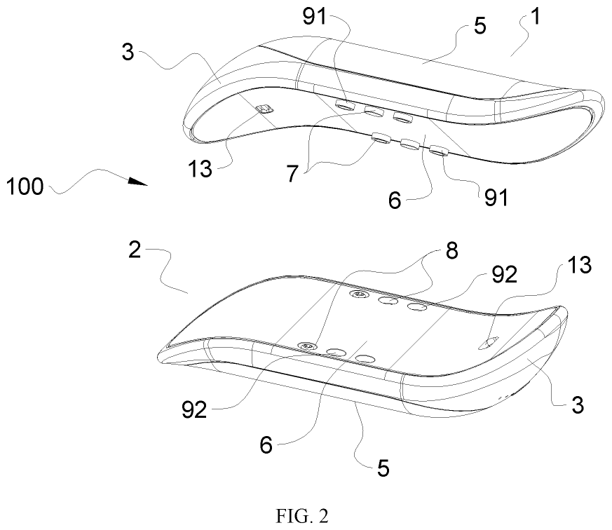

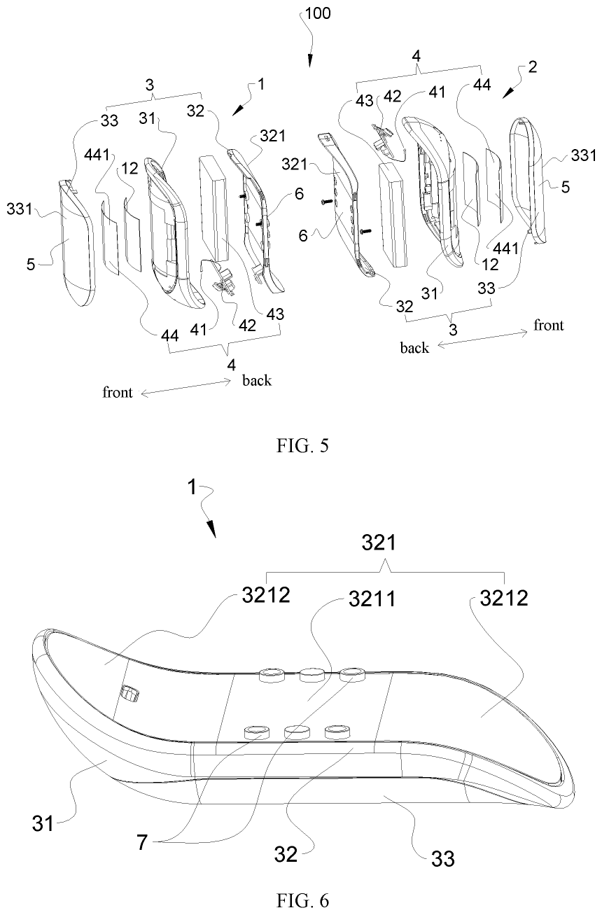

Referring to FIG. 2 and FIG. 5, each of the first body 1 and the second body 2 includes the housing 3 and the heat generating unit 4 received in the housing 3, and the heating surface 5 and the contacting surface 6 are respectively arranged on the outer surface of the housing 3 of each of the first and second bodies 1, 2. In an embodiment of the present disclosure, the heating surface 5 of the housing 3 is opposite to the contacting surface 6. It can be understood that in other embodiments, the heating surface 5 can be adjacent to the contacting surface 6, such as the heating surface 5 arranged on a front side of the housing 3, and the contacting surface 6 arranged on a left side or a right side of the housing 3 that is adjacent to the front side of the housing 3.

The housing 3 of the hand warmer 100 of the present disclosure is a long-strip-shaped structure, and the heating surface 5 of the housing 3 is in a curved shape suitable for being gripped by a hand. Referring to FIG. 1 and FIG. 2, the housing 3 is a long-strip structure that means that a width of the housing 3 is narrower and a length is longer, which makes the hand warmer 100 be easily gripped when the hand warmer 100 is used separately or in combination. At the same time, the heating surface 5 of each of the first body 1 and the second body 2 of the present disclosure is in a curved shape that is suitable for being gripped by hands, and the curved shape is easy to be gripped and fits a center curvature of a palm of the hand.

Referring to FIG. 2, the first body 1 includes a plurality of first inserting members 7 arranged on the contacting surface 6 of the housing 3, and the second body 2 includes a plurality of second inserting members 8 arranged on the contacting surface 6 of the housing 3 and correspondingly connected to the plurality of first inserting members 7. After the plurality of first inserting members 7 is engaged with the plurality of second inserting members 8, correspondingly, both the first body 1 and the second body 2 are combined into a whole; referring to FIG. 1, at this time, the first inserting member 7 of the first body 1 is connected to the second inserting member 8 of the second body 2, and the contacting surface 6 of the first body 1 is adhered to the contacting surface 6 of the second body 2, so that both the first body 1 and the second body 2 are combined into a whole. In this way, the first body 1 and the second body 2 are fixed together to be taken as an integral product, which can be configured to be held by one hand for warming the hand.

When the hand warmer 100 of the present disclosure is in a combination state, the hand warmer 100 of the present disclosure that is held by one hand has the following advantages:
the combined hand warmer 100 has two heating surfaces 5 for using be a single hand, that is, the combined product has both the heating surface 5 of the first body 1 and the heating surface 5 of the second body 2 for the single hand, thereby increasing a heating area for the single hand, and improving a heating effect during being held by the single hand.

After the hand warmer 100 of the present disclosure is combined, the first body 1 and the second body 2 can be separated from a connection position between the first inserting member 7 and the second inserting member 8. That is, a connection structure between the first body 1 and the second body 2 of the present disclosure is a quick installation and quick disassembly structure, which can conveniently combine and separate the first body 1 and the second body 2.

The hand warmer 100 of the present disclosure can be used as a whole by one hand after combining the first and second bodies 1, 2, or can use one of the first and second bodies 1, 2 by one hand after separating the first and second bodies 1, 2. That is, the first and second bodies 1, 2 that have been combined can be held to be used by one hand, or separated can be held to be used by one hand, so as to adapt for different usage scenarios and requirements. The first body 1 and the second body 2 are fixed as a whole after being combined, which is conveniently received and carried.

The first inserting member 7 of the present disclosure is an inserting post 91 protruding outwardly from the contacting surface 6, and the second inserting member 8 is a hole 92 concavely arranged on the contacting surface 6 and engaged with the first inserting member 7; or the first inserting member 7 is a hole 92 concavely arranged on the contacting surface 6, and the second inserting member 8 is an inserting post 91 protruding outwardly from the contacting surface 6 and engaged with the first inserting member 7.

That is, a plug connection structure between the first body 1 and the second body 2 of the present disclosure adopts a hole and a post connected to the hole by plugging with the hole and the post. As an embodiment of the present disclosure, referring to FIG. 2, the first inserting member 7 of the first body 1 is the inserting post 91 protruding outwardly from the contacting surface 6, and the second inserting member 8 of the second body 2 is the hole 92 concavely arranged on the contacting surface 6 and engaged with the first inserting member 7. The inserting post 91 of the contacting surface 6 of the first body 1 is inserted into the hole 92 of the contacting surface 6 of the second body 2 to complete an insertion connection of the first body 1 and the second body 2. Preferably, as shown in FIG. 2, the contacting surface 6 of the first body 1 is symmetrically arranged with a plurality of inserting posts 91 in two rows, such as three inserting posts 91 arranged in each row of the two rows, so that there are six inserting posts 91 in total. The contacting surface 6 of the second body 2 is symmetrically arranged with a plurality of holes 92 in two rows, such as three holes 92 arranged in each row of the two rows, so that there are six holes 92 in total. A connection of the plurality of holes 92 and the plurality of posts 91 can enhance connection stability of the first body 1 and the second body 2. It can be understood that in other embodiments of the present disclosure, both the plurality of inserting posts 91 and the plurality of holes 92 can be set on the contacting surface 6 of the first body 1. Correspondingly, the plurality of holes 92 can be set on the contacting surface 6 of the second body 2 to connect with and the plurality of inserting posts 91 of the contacting surface 6 of the first body 1; at the same time, the plurality of inserting posts 91 can be arranged on the contacting surface 6 of the second body 2 to connect with the plurality of holes 92 of the contacting surface 6 of the first body 1.

The insertion connection structure between the first body 1 and the second body 2 of the present disclosure by engaging the hole with the post is simple in manufacturing and processing, and has a low cost thereof.

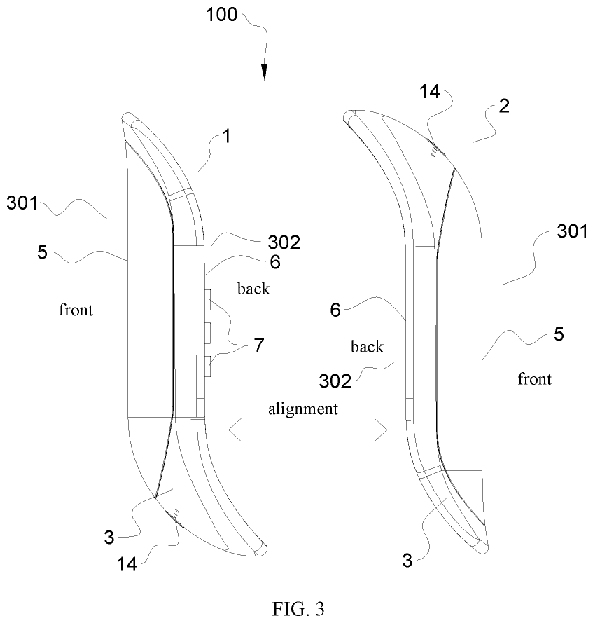

Preferably, referring to FIG. 3, a front side surface 301 of the housing 3 of the hand warmer 100 of the present disclosure is the heating surface 5, and a back side surface 302 of the housing 3 is the contacting surface 6. In an embodiment of the present disclosure, a surface that comes into contact with hands and generates heat during using the hand warmer 100 is defined as the front side surface 301 of the housing 3, while a surface that brings the first body 1 close to the second body 2 after combining the hand warmer 100 is defined as the back side surface 302 of the housing 3. As shown in FIG. 3, a left side of the first body 1 is defined as the front side surface 301 of the housing 3 of the first body 1, and a right side of the first body 1 is defined as the back side surface 302 of the housing 3 of the first body 1; the left side of the second body 2 is defined as the back side surface 302 of the housing 3 of the second body 2, and the right side of the second body 2 is defined as the front side surface 301 of the housing 3 of the second body 2.

The contacting surface 6 of the first body 1 is aligned with the contacting surface 6 of the second body 2 and then and pressed onto the contacting surface 6 of the second body 2, the first inserting member 7 is engaged with the second inserting member 8; that is, after aligning and pressing the back side surface 302 of the first body 1 with the back side surface 302 of the second body 2, the first inserting member 7 of the first body 1 is connected to the second inserting member 8 of the second body 2 through a plug connection way. The contacting surface 6 of the first body 1 is aligned with the contacting surface 6 of the second body 2 of the present disclosure means that a complete alignment of edges of the two contacting surfaces 6 of the first and second bodies 1, 2. After the two contacting surfaces 6 are aligned with each other, the first inserting member 7 on the contacting surface 6 of the first body 1 is also aligned with the second inserting member 8 on the contacting surface 6 of the second body 2; and then, the first body 1 and the second body 2 press towards each other to complete the insertion connection therebetween. After aligning the contacting surface 6 of the first body 1 with the contacting surface 6 of the second body 2, the combination can be completed by pressing with one hand, that is, the combination operation of the first body 1 and the second body 2 of the present disclosure is simple and fast.

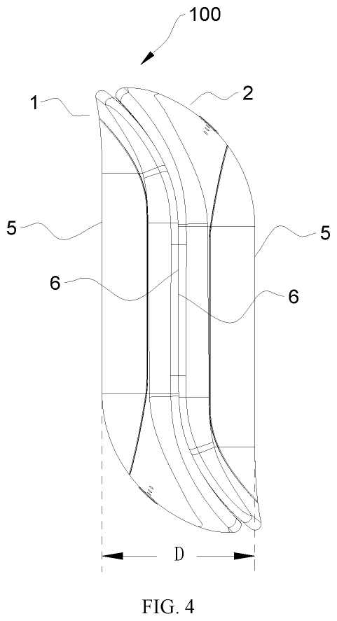

Referring to FIG. 4, after the first body 1 and the second body 2 of the hand warmer 100 of the present disclosure are combined together, the contacting surface 6 of the first body 1 is adhered to the contacting surface 6 of the second body 2. That is, after the first body 1 and the second body 2 are combined with each other, the contacting surfaces 6 of the first body 1 and the second body 2 completely fit with each other without leaving any gaps between the first body 1 and the second body 2, which can reduce a thickness of the combined hand warmer 100 to be conveniently gripped by one hand.

The heating surface 5 and the contacting surface 6 of the present disclosure are respectively arranged on the front side surface 301 and the back side surface 302 of the housing 3, and the heating surface 5 of the housing 3 is opposite to the contacting surface 6 of the housing 3, so that both the first body 1 and the second body 2 have the following advantages after the first body 1 and the second body 2 are combined with each other:

referring to FIG. 4, the combined hand warmer 100 has two heating surfaces 5 on both sides thereof for using by one hand. In this way, when holding the combined hand warmer 100 as shown in FIG. 4, a hand palm can fit onto the heating surface 5 of the first body 1 on the left, and fingers can fit onto the heating surface 5 of the second body 2 on the right, so that both the palm and the fingers of the single hand can be warmed simultaneously. While, for a non-combined hand warmer, when holding the first body 1 or the second body 2 by one hand, and when the hand palm is in contact with the heating surface 5 of the housing 3, the finger is in contact with the contacting surface 6 of the housing 3 that doesn't generate heat, in this way, the combined hand warmer 100 of the present disclosure has a better heating effect by holding the combined hand warmer 100 with one hand than by holding a non-combined type hand warmer with one hand.

Furthermore, referring to FIG. 4, a range of a total thickness D of the first body 1 and the second body 2 of the present disclosure is 20-40 mm after the first body 1 is engaged with the second body 2. The total thickness D that the first body 1 is combined with the second body 2 is a distance from the leftmost point on the heating surface 5 of the first body 1 to the rightmost point on the heating surface 5 of the second body 2 as shown in FIG. 4. In the embodiment of the present disclosure, the range of the total thickness D after the first body 1 is combined with the second body 2 is 20-40 mm, which makes the combined hand warmer 100 relatively thin and easy to be gripped.

Specifically, referring to FIG. 5, the housing 3 includes a front housing 31, a back housing 32 arranged behind the front housing 31 and connected to the front housing 31, and a heat conducting shell 33 arranged in front of the front housing 31 and connected to the front housing 31. A front side surface 331 of the heat conducting shell 33 is the heating surface 5, and a back side surface 321 of the back housing 32 is the contacting surface 6. The heat conducting shell 33 of the present disclosure can be metal materials with good thermal conductivity, such as aluminum or aluminum alloy or copper. The front housing 31 and the back housing 32 can be made of high-temperature resistant plastic materials, and the front housing 31 and the back housing 32 cooperatively form a cavity therebetween to receive components of the heat generating unit 4. The heat conducting shell 33, the front housing 31 and the back housing 32 can be connected to each other by a buckle way, a snap way, or an adhesive way.

Specifically, the heat generating unit 4 includes a control circuit board 42, a battery 43, a heat generating member 44 and a temperature sensor 41. The battery 43, the heat generating member 44 and the temperature sensor 41 are electrically connected to the control circuit board 42, respectively. Both the control circuit board 42 and the battery 43 are arranged between the front housing 31 and the back housing 32, and both the temperature sensor 41 and the heat generating member 44 are installed between the front housing 31 and the heat conducting shell 33 to abut against the heat conducting shell 33, respectively.

The control circuit board 42 is equipped with a control circuit, such as a microcontroller or other control chips, to control all the battery 43, the heat generating member 44 and the temperature sensor 41 to work. For example, the control circuit board 42 is configured to control for charging and discharging the battery 43, control the on-off and power adjustment of the heat generating member 44, and receive temperature signals detected by the temperature sensor 41. The battery 43 is configured to supply power to the control circuit board 42, the heat generating member 44, and the temperature sensor 41. The heat generating member 44 generates heat after being energized, as shown in FIG. 5. Due to the heat generating member 44 abutting against an inner side of the heat conducting shell 33, so that the heat generated by the heat generating member 44 is transmitted to the heat conducting shell 33, causing a temperature of the heat conducting shell 33 to increase for warming hands. The temperature sensor 41 is abutted against the inner side of the heat conducting shell 33 and electrically connected to the control circuit board 42 through wires, to detect the temperature of the heat conducting shell 33. When the temperature of the heat conducting shell 33 that is detected by the temperature sensor 41 reaches a preset temperature, the control circuit board 42 will control the heat generating member 44 to stop working based on temperature signals feedback from the temperature sensor 41, so as to prevent overheating of the heat conducting shell 33 from scalding hands, and ensure safe usage. When the temperature of the heat conducting shell 33 is lower than the preset temperature, the control circuit board 42 controls the heat generating member 44 to start working, to maintain the temperature of the heat conducting shell 33 within a constant range.

As an embodiment of the present disclosure, referring to FIG. 6, a middle part 3211 of the back side surface 321 of the back housing 32 is a plane, and two ends 3212 of the back side surface 321 of the back housing 32 are curved surfaces. Similarly, the middle part 3211 of the back side surface 321 of the back housing 32 of the second body 2 is also a plane, and two ends 3212 of the back side surface 321 of the back housing 32 of the second body 2 are also curved surfaces. The middle part 3211 of the back side surface 321 of the back housing 32 is a plane configured to arrange the first inserting member 7 or the second inserting member 8, so as to conveniently combine the first body 1 and the second body 2 together. The two ends 3212 of the back side surface 321 of the back housing 32 are set as curved surfaces, on the one hand, the combined product is more beautiful, on the other hand, the two ends of the combined product that are curved surfaces can be gripped better, on the third hand, when the first body 1 and the second body 2 are combined, they must be aligned with each other according to a fixed position to be combined. Referring to FIG. 3, a control switch 14 is arranged on a lower end of the first body 1, and the control switch 14 is also arranged on an upper end of the second body 2. When the control switch 14 of the first body 1 is arranged on the lower end of the first body 1, and the control switch 14 of the second body 2 is arranged on the upper end of the second body 2, the first body 1 and the second body 2 can be aligned and combined with each other. If the control switch 14 of the first body 1 is arranged on the upper end of the first body 1, and the control switch 14 of the second body 2 is also arranged on the upper end of the second body 2, the first body 1 and the second body 2 can't be combined with each other. Therefore, such curved surface design plays a role in positioning and alignment of the first body 1 and the second body 2.

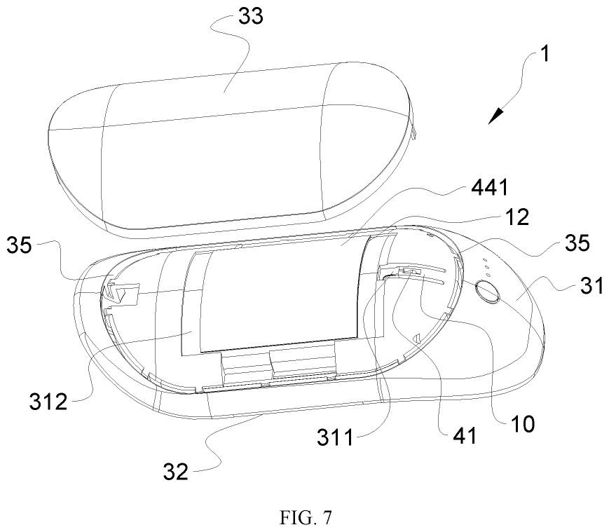

Referring to FIG. 7, a fixing member 10 is arranged on the front housing 31 or the heat conducting shell 33 and configured to fix the temperature sensor 41 and be in contact with the heat conducting shell 33. In this way, during using the hand warmer 100 of the present disclosure, the fixing member 10 is provided that the temperature sensor 41 can always abut against the heat conducting shell 33.

In a conventional technology, the temperature sensor 41 is in contact with the heat conducting shell 33 by wires that are connected to the temperature sensor 41 extending a certain length to abut against the heat conducting shell 33, rather than providing a structure to adhere or fix the temperature sensor 41 with the heat conducting shell 33, in this way, the temperature sensor 41 is prone to loosening and coming out of contact with the heat conducting shell 33 during using the hand warmer 100, resulting in inaccurate temperature measurement. If an actual temperature of the heat conducting shell 33 exceeds the preset temperature, but a distance between the temperature sensor 41 and the heat conducting shell 33 is great due to disconnect the temperature sensor 41 and the heat conducting shell 33, the temperature detected by the temperature sensor 41 will be lower than the actual temperature of the heat conducting shell 33, which can cause burns and other safety hazards. The fixing member 10 of the present disclosure is provided to fix the temperature sensor 41 and keeps the temperature sensor 41 in contact with the heat conducting shell 33; in this way, even if the product shakes during using the hand warmer 100, the temperature sensor 41 is always in contact with the heat conducting shell 33, thereby ensuring an accuracy of temperature measurement and further improving the safety of the product.

Preferably, the heat generating member 44 of the present disclosure is a PI flexible electrothermal film 441 attached to the heat conducting shell 33. The PI flexible electrothermal film 441 is a sheet-shaped flexible material that can adapt to a curvature of the heat conducting shell 33. The PI flexible electrothermal film 441 is made through a high temperature and high pressure heating way by taking a polyimide film as an external insulator thereof, and taking a metal foil and a metal wire as an internal conductive heating body thereof, which can be bent adaptively, with good insulation and high heating efficiency.

Referring to FIG. 7, both the temperature sensor 41 and the PI flexible electrothermal film 441 of the present disclosure abut against the inner side of the heat conducting shell 33, and the PI flexible electrothermal film 441 can be glued onto the inner side of the heat conducting shell 33. As an implementation method, the fixing member 10 of the present disclosure is arranged on the front housing 31, so that the temperature sensor 41 is fixed on the front housing 31 and is in contact with the inner side of the heat conducting shell 33.

Furthermore, referring to FIG. 7, a high-temperature resistant foam 12 is arranged between the PI flexible electrothermal film 441 and the front housing 31; that is, in FIG. 7, the high-temperature resistant foam 12 is arranged on a lower part of the PI flexible electrothermal film 441, and a lower part of the high-temperature resistant insulation foam 12 is attached to the front housing 31. The high-temperature resistant foam 12 is made of high-temperature resistant materials such as EVA. On the one hand, the high-temperature resistant foam 12 is configured to preserve heat generated by the PI flexible electrothermal film 441 to prevent heat from escaping from the front housing 31, and on the other hand, the high-temperature resistant foam 12 can perform heat insulation on the front housing 31 to prevent the heat from transmitting to the front housing 31. The high-temperature resistant foam 12 can be coated on both sides, with one side pasting and fixing the PI flexible electrothermal film 441 and the other side pasting and fixing on the front housing 31.

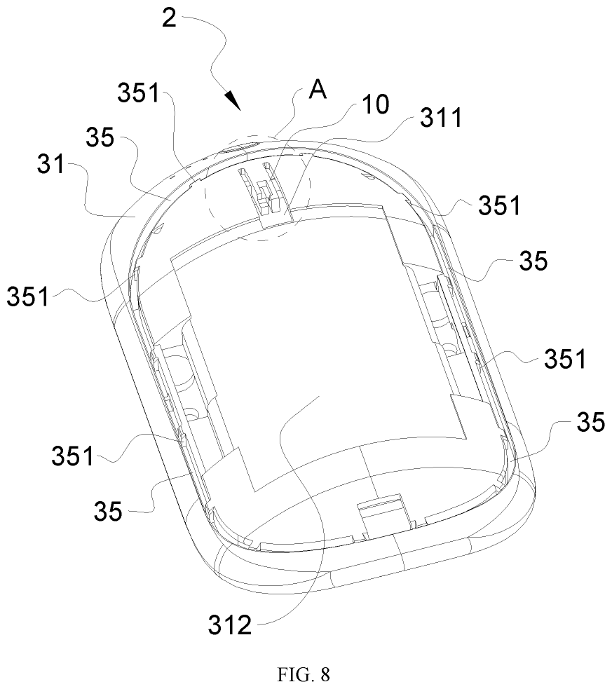

Preferably, referring to FIG. 7 and FIG. 8, the front housings 31 of the first and second bodies 1, 2 are also recessed with installation windows 312 for installing the high-temperature resistant foam 12 therein.

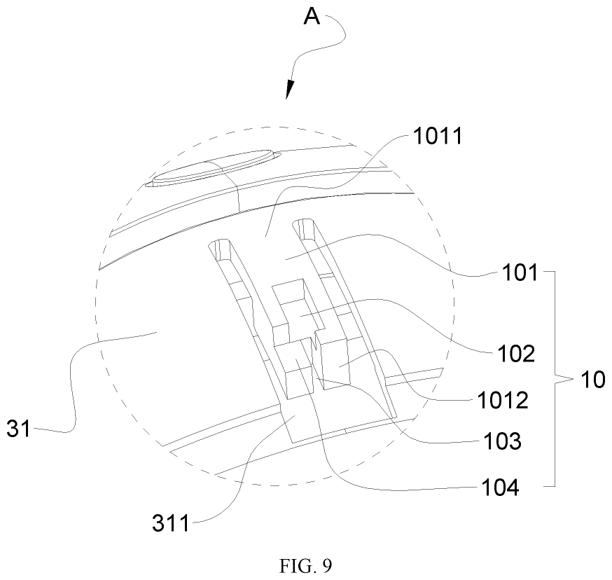

Specifically, referring to FIG. 9, the front housing 31 of each of the first and second bodies 1, 2 includes a wiring window 311 passing through the front housing 31 from front and back. The fixing member 10 includes a rod 101, a clamping groove 102 arranged on the rod 101 and facing the heat conducting shell 33, and a wiring groove 103 connected with the clamping groove 102 and passing through the rod 101 from front and back. The rod 101 includes a first end 1011 connected to the front housing 31, and a second end 1012 opposite to the first end 1011 and suspended inside the wiring window 311.

The temperature sensor 41 is connected to the control circuit board 42 through wires, and the control circuit board 42 is arranged between the front housing 31 and the back housing 32, while the heat generating member 44 is arranged between the front housing 31 and the heat conducting shell 33. The temperature sensor 41 is installed on the fixing member 10 of the front housing 31 and connected to the heat conducting shell 33. Therefore, the wires connected between the temperature sensor 41 and the control circuit board 42 needs to pass through the front housing 31. The wiring window 311 that is arranged on the front housing 31 is configured to pass through the wires, while the clamping groove 102 is configured to clamp and fix the temperature sensor 41, so that the temperature sensor 41 can expose out of a certain height to abut against the heat conducting shell 33. The wiring groove 103 is configured to receive the wires that pass through the wiring window 311.

Furthermore, an installation portion 104 is arranged on a surface 1013 of the rod 101 towards the heat conducting shell 33, and connected to both the clamping groove 102 and the wiring groove 103. The installation portion 104 is provided to conveniently operate objects that are received in both the clamping groove 102 and the wiring groove 103 to be pressed, moved and clamped.

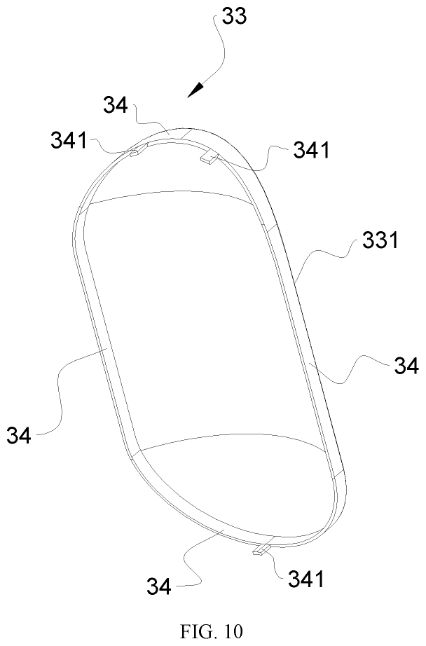

Referring to FIG. 10, a flange 34 is arranged on an edge 332 of the heat conducting shell 33, and referring to FIG. 7 and FIG. 8, a snapping groove 35 is arranged on a surface of the front housing 31 facing the heat conducting shell 33 and clamped with the flange 34. In this way, the heat conducting shell 33 of the present disclosure can be very easy to install on the front housing 31. Aligning the flange 34 of the heat conducting shell 33 with the snapping groove 35 of the front housing 31, and then pressing the heat conducting shell 33 to insert the flange 34 of the heat conducting shell 33 into the clamping groove 35 of the front housing 31. The flange 34 is tightly matched with the clamping groove 35 to fix the heat conducting shell 33 on the front housing 31. When pressing the heat conducting shell 33, the heat conducting shell 33 itself also undergoes deformation, so that the flange 34 also has a tension to engage with a sidewall of the clamping groove 35.

In the conventional technology, a plurality of flexible clamping strips is generally set at edges of the heat conducting shell 33, and a plurality of clamping holes is arranged on the front housing 31. During installing the front housing 31 and the heat conducting shell 33, the plurality of clamping strips of the heat conducting shell 33 are correspondingly inserted into the plurality of clamping holes of the front housing 31, and then the plurality of clamping strips is bent one by one to fasten the heat conducting shell 33 onto the front housing 31. In this way, when installing the heat conducting shell 33, it is necessary to manually bend each of the plurality of clamping strips in sequence, which is very inconvenient, takes a long time to install, and has low efficiency. At the same time, the plurality of clamping strips is prone to being broken after performing a plurality of bends, which can easily cause to damage the plurality of clamping strips of the heat conducting shell 33 when repairing and disassembling heat conducting components within the hand warmer. While, the present disclosure provides the flange 34 that is engaged with the snapping groove 35 to install the heat conducting shell 33 and the front housing 31, which is very fast and efficient. The assembly of the heat conducting shell 33 and the front housing 31 can be completed by aligning the flange 34 of the heat conducting shell 33 with the snapping groove 35 of the front housing 31, and then pressing the heat conducting shell 33 to engage the flange 34 with the snapping groove 35, so as to further completely install the heat conducting shell 33 and the front housing 31.

Preferably, referring to FIG. 8, a plurality of protrusions 351 is spaced in the clamping groove 35 of the front housing 31 and abutting against the flange 34. In this way, when the flange 34 of the heat conducting shell 33 is embedded into the clamping groove 35 of the front housing 31, the plurality of protrusions 351 abuts against a sidewall of the flange 34, thereby improving a clamping effect between the front housing 31 and the heat conducting shell 33.

Furthermore, a clamping strip 341 is arranged on the flange 34 of the heat conducting shell 33, correspondingly, a clamping hole is also set on the front housing 31 to insert the clamping strip 341 therein, and then the clamping strip 341 is bent to obtain a buckle connection between the front housing 31 and the heat conducting shell 33.

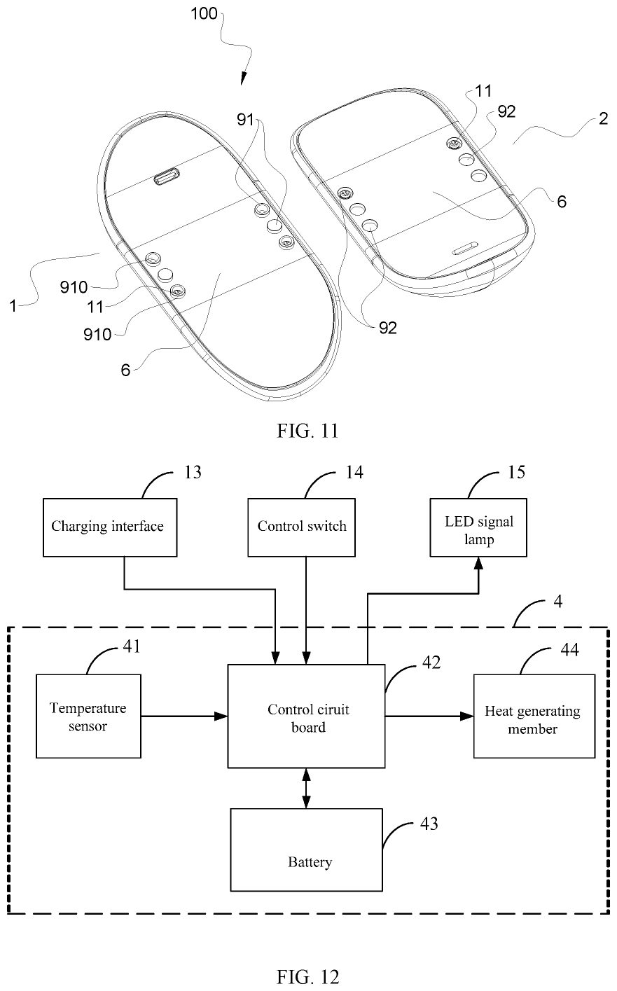

Preferably, referring to FIG. 11, a screw 11 is hidden in the hole 92 of the contacting surface 6. The inserting post 91 of the contacting surface 6 is a tubular post 910, and the screw 11 is hidden and arranged inside the tubular post 910. In this way, the front housing 31 and the back housing 32 can be connected with each other through the screw 11 to ensure connection firmness between the housing 31 and the back housing 32. At the same time, the screw 11 can be hidden in the hole 92 or hidden in the tubular post 910 to avoid the screw 11 protruding from the outer surface of the housing 3 and affecting an aesthetics thereof. On the one hand, the tubular post 910 can receive the screw 11 therein, and on the other hand, when the screw 11 isn't received in the tubular post 910, the tubular post 910 can be connected to the hole 92 that receives the screw 11 therein.

Referring to FIG. 1, FIG. 2 and FIG. 12, preferably, a charging interface 13 is arranged on the contacting surface 6 of the housing 3 and electrically connected with the heat generating circuit 4. The charging interface 13 is configured to connect to an external power source for charging the battery 43. The charging interface 13 can be a USB interface or a TYPE-C interface, etc. The charging interface 13 of the present disclosure is arranged on the contacting surface 6 of the housing 3, so that when the hand warmer 100 is in the combination state, the charging interface 13 an be hidden.

Preferably, the housing 3 is further provided with a control switch 14 electrically connected with the heat generating circuit 4 and configured to control to start and stop the heat generating member 44.

Preferably, the housing 3 is further provided with an LED signal lamp 15 electrically connected with the heat generating circuit 4. The LED signal lamp 15 can be a combination of a plurality of LEDs, such as red LEDs, green LEDs, and yellow LEDs, configured to indicate a working status, a charging status, or a battery level status, etc.

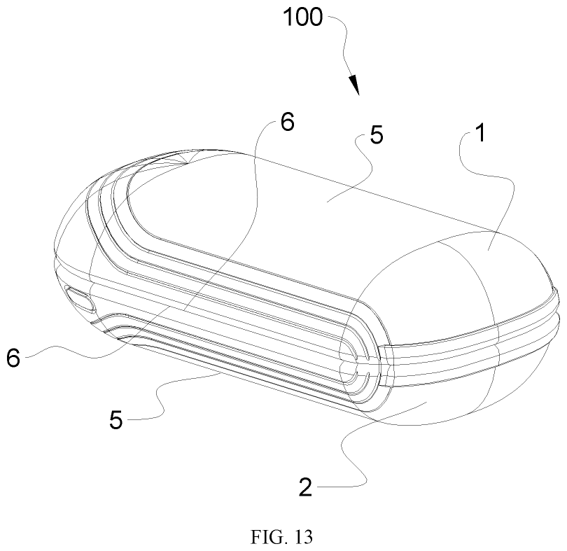

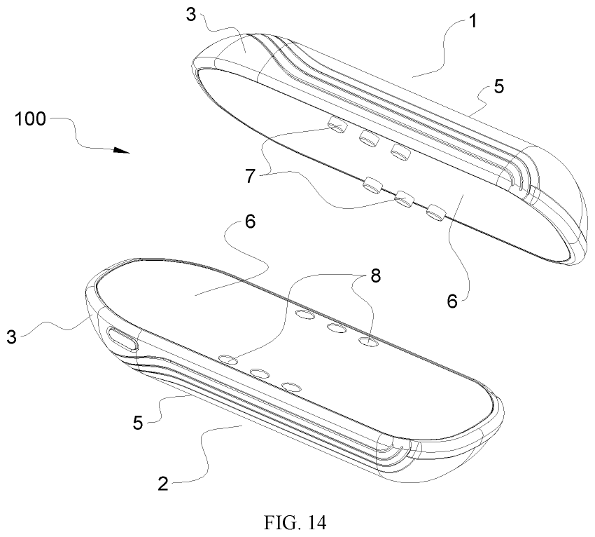

Referring to FIG. 13 and FIG. 14, a hand warmer 100 that can be combined or used separately in accordance with a second embodiment of the present disclosure is provided. A difference between the hand warmer 100 of the second embodiment and the hand warmer 100 of the first embodiment is that: the back side surface 321 of the back housing 32 of the second embodiment is a plane. That is, as shown in FIG. 13 and FIG. 14, the middle part 3211 of the back side surface 321 of each of the first and second bodies 1, 2 is a plane, and the two ends 3212 are also planes, so that the back side surface 321 of the back housing 32 is a whole plane. In this way, when the first body 1 is combined with the second body 2, there is no need to distinguish upper and lower positions of the first body 1 and the second body 2 to be aligned. Other structures and principles of the hand warmer 100 of the second embodiment are the same as that of the hand warmer 100 of the first embodiment, which will not be repeated here.

The hand warmer 100 of the above embodiments of the present disclosure can be combined or used separately. By setting the whole product into the first body 1 and the second body 2 that can independently generate heat, each of the first body 1 and the second body 2 includes the heating surface 5 and the contacting surface 6, the first body 1 is equipped with the first inserting member 7, and the second body 2 is equipped with the second inserting member 8; when the first inserting member 7 and the second inserting member 8 are connected with each other, the first body 1 and the second body 2 are combined together; at this time, the combined product can be held and used by one hand. When a user needs to warm left and right hands separately, separating the first body 1 from the second body 2 that have been combined. The first body 1 and the second body 2 of the hand warmer 100 of the present disclosure can be combined or used separately to meet different requirements of users, and the combined hand warmer 100 is conveniently to be received and carried.

Although the features and members of the present disclosure are described as embodiments in particular combinations, each feature or member can be used alone or in other various combinations within the principles of the present disclosure to the full extent indicated by the broad general meaning of the terms in which the appended claims are expressed.

What is claimed is:

1. A hand warmer that can be combined or used separately comprising:
a first body and a second body with the same structure as the first body, and mutually combined with the first body; wherein
each of the first body and the second body comprises a long-strip-shaped housing and a heat generating unit received in the housing, the housing comprising a heating surface arranged on an outer surface thereof, and a contacting surface opposite to the heating surface; and wherein the heat generating unit is configured to generate heat and then transmit the heat to the heating surface of the housing, and the heating surface of the housing is in a curved shape suitable for being gripped by a hand;
the first body comprising a plurality of first inserting members arranged on the contacting surface of the housing;
the second body comprising a plurality of second inserting members arranged on the contacting surface of the housing and correspondingly connected to the plurality of first inserting members; and wherein
after the plurality of first inserting members is engaged with the plurality of second inserting members, correspondingly, both the first body and the second body are combined into a whole, and the first body and the second body can be separated from a connection position between the first body and the second body.

2. The hand warmer as claimed in claim 1, wherein the first inserting member is an inserting post protruding outwardly from the contacting surface, and the second inserting member is a hole concavely arranged on the contacting surface and engaged with the first inserting member; or the first inserting member is a hole concavely arranged on the contacting surface, and the second inserting member is an inserting post protruding outwardly from the contacting surface and engaged with the first inserting member.

3. The hand warmer as claimed in claim 1, wherein a front side surface of the housing is the heating surface, and a back side surface of the housing is the contacting surface; after aligning and pressing the contacting surface of the first body with the contacting surface of the second body, the plurality of first inserting members is connected to the plurality of second inserting members in a corresponding manner, and the contacting surface of the first body is in contact with the contacting surface of the second body.

4. The hand warmer as claimed in claim 1, wherein a range of a total thickness of the first body and the second body is 20-40 mm after the first body is engaged with the second body.

5. The hand warmer as claimed in claim 1, wherein the housing comprises a front housing, a back housing arranged behind the front housing and connected to the front housing, and a heat conducting shell arranged in front of the front housing and connected to the front housing, a front side surface of the heat conducting shell is the heating surface, and a back side surface of the back housing is the contacting surface.

6. The hand warmer as claimed in claim 5, wherein the back side surface of the back housing is a plane.

7. The hand warmer as claimed in claim 5, wherein a middle part of the back side surface of the back housing is a plane, and two ends of the back side surface of the back housing are curved surfaces.

8. The hand warmer as claimed in claim 5, wherein the heat generating unit comprises a temperature sensor, and a fixing member is arranged on the front housing or the heat conducting shell and configured to fix with the temperature sensor and be in contact with the heat conducting shell.

9. The hand warmer as claimed in claim 8, wherein the front housing comprises a wiring window passing through the front housing from front and back, and the fixing member comprises a rod, a clamping groove arranged on the rod and facing the heat conducting shell, and a wiring groove connected with the clamping groove and passing through the rod from front and back; the rod comprising a first end connected to the front housing, and a second end opposite to the first end and suspended inside the wiring window.

10. The hand warmer as claimed in claim 9, wherein an installation portion is arranged on a surface of the rod towards the heat conducting shell, and connected to both the clamping groove and the wiring groove.

11. The hand warmer as claimed in claim 5, wherein a flange is arranged on an edge of the heat conducting shell, and a snapping groove is arranged on a surface of the front housing facing the heat conducting shell and clamped with the flange.

12. The hand warmer as claimed in claim 11, wherein a plurality of protrusions is spaced in the clamping groove of the front housing and abutting against the flange.

13. The hand warmer as claimed in claim 2, wherein a screw is hidden in the hole of the contacting surface; and/or
the inserting post on the contacting surface is a tubular post, and the screw is hidden and arranged inside the tubular post.

14. The hand warmer as claimed in claim 8, wherein the heat generating unit comprises a control circuit board electrically connected to the temperature sensor, a battery and a heat generating member electrically connected to the control circuit board, respectively; both the control circuit board and the battery arranged between the front housing and the back housing, and the heat generating member installed between the front housing and the heat conducting shell and being in contact with the heat conducting shell.

15. The hand warmer as claimed in claim 14, wherein the heat generating member is a PI flexible electrothermal film attached to the heat conducting shell.

16. The hand warmer as claimed in claim 15, wherein a high-temperature resistant foam is arranged between the PI flexible electrothermal film and the front housing.

17. The hand warmer as claimed in claim 1, wherein a charging interface is arranged on the contacting surface of the housing and electrically connected with the heat generating circuit.

18. The hand warmer as claimed in claim 1, wherein the housing is further provided with a control switch electrically connected with the heat generating circuit.

19. The hand warmer as claimed in claim 1, wherein the housing is further provided with an LED signal lamp electrically connected with the heat generating circuit.
